SEQUENCE LISTING (3 sequences)

SEQ ID NO: 1 - DNA, 20 bp, Artificial Sequence
  Synthetic Primer
cggtgtgagaagtgcagcaa

SEQ ID NO: 2 - DNA, 19 bp, Artificial Sequence
  Synthetic Primer
cctctcgcaagtgctccat

SEQ ID NO: 3 - DNA, 24 bp, Artificial Sequence
  Synthetic Primer
ccagaccatagcacactcgggcac

(19) United States
(12) Patent Application Publication
(10) Pub. No.: US 2010/0151463 A1
(43) Pub. Date: Jun. 17, 2010

(54) Method for Determining the Likelihood of Response to HER2 Inhibitors

(76) Inventors: Frederick L. Baehner, San Francisco, CA (US); Drew Watson, Los Altos, CA (US); Tara Maddala, Redwood City, CA (US); Steve Shak, Hillsborough, CA (US); Robert Gray, Boston, MA (US); Joseph Sparano, Pleasantville, NY (US)

Correspondence Address: Genomic Health, Inc.;c/o Kathleen Determann, 301 Penobscot Road, Redwood City, CA, 94063, US

(21) Appl. No.: 12/554,783

(22) Filed: Sep. 4, 2009

Related U.S. Application Data
(60) Provisional application No. 61/094,827, filed on Sep. 5, 2008.

(51) Int. Cl. C12Q 1/68

(52) U.S. Cl. 435/6

(57) ABSTRACT

The present disclosure provides methods for determining the HER2 Status of a cancer tumor sample.

### CROSS-REFERENCE TO RELATED APPLICATIONS

This application claims priority benefit to U.S. provisional application Ser. No. 61/094,827, filed Sep. 5, 2008, which application is incorporated herein in its entirety.

### INTRODUCTION

Invasive breast cancer is the second most common form of cancer and leading cause of cancer deaths among women in the United States. In developed countries, the lifetime risk for breast carcinoma is over 13%. (Pfeifer JD Molecular Genetic Testing in Surgical Pathology. Philadelphia, Pa., Lippincott Williams & Wilkens, 2006: 401-414).

The proto-oncogene HER2 (also known as HER2/neu or ERBB2) is a member of the ERBB family of cell surface receptors, which regulate cellular processes implicated in tumor growth, including proliferation and differentiation. Certain aspects of this disclosure relate to a method for identifying which breast cancer patients are more likely to benefit from treatment using chemotherapeutic agents that target HER2.

### SUMMARY

In one aspect, this disclosure provides methods for determining the HER2 Status of a sample by measuring a HER2 expression level from a cancer tumor sample using quantitative reverse transcriptase polymerase chain reaction (QRT-PCR), normalizing the HER2 expression level relative to at least one reference gene to generate a normalized HER2 expression level, comparing the normalized HER2 expression level to predetermined HER2 expression cutpoints to determine a HER2 Status, wherein predetermined HER2 expression cutpoints are based on a validated test, wherein the HER2 Status of the cancer tumor is one of the classifications consisting of positive, negative, and equivocal, and wherein the likelihood that the sample is classified as HER2-equivocal is negatively optimized. In some embodiments, the cancer tumor is a breast cancer tumor.

In some embodiments, negatively optimized comprises establishing the HER2 expression cutpoints such that the likelihood that the sample is classified as HER2-equivocal is one from the list consisting of less than about 5%, less than about 7.5%, less than about 10%, and less than about 17%.

In some embodiments, the predetermined HER2 expression cutpoints comprise an upper expression cutpoint and a lower expression cutpoint. In some embodiments, the predetermined HER2 expression cutpoints are an upper expression cutpoint and a lower expression cutpoint. In some embodiments, the predetermined HER2 expression cutpoints are established such that the concordance rate between HER2 Status by QRT-PCR and HER2 Status by IHC is high when measured in a statistically significant number of cancer tumors.

In some embodiments, the predetermined HER2 expression cutpoints are based upon ER status of the sample. In some embodiments, the ER status is determined by QRT-PCR.

In some embodiments, the concordance rate between HER2 Status by QRT-PCR and HER2 Status by IHC is one from the list consisting of: greater than about 95%, greater than about 92.5%, and greater than about 90%.

In some embodiments, the HER2 expression level is normalized relative to expression levels of ACTB, GAPDH, GUSB, RPLP0 and TFRC.

In some embodiments the HER2 expression level is measured using a HER2 expression product. In some embodiments, the HER2 expression product is RNA.

In some embodiments, the normalized HER2 expression level is calculated as Standard Normalized Expression level. In some embodiments, the HER2 expression product is measured using QRT-PCR probe/primer sets targeted to intronic HER2 sequences.

In some embodiments the sample is fixed paraffin embedded tissue.

This disclosure further provides methods for identifying patients for treatment with a HER2 using a minimally equivocal HER2 Status of a tumor sample obtained from a cancer patient by measuring the expression level of a HER2 expression product in the tumor sample, normalizing the HER2 expression level relative to at least one reference gene, comparing the normalized expression level to predetermined HER2 cutpoints, including at least a lower HER2 expression cutoff and an upper HER2 expression cutoff, and classifying the tumor according to HER2 Status, wherein a HER2-positive Status indicates that the patient is a candidate for treatment with a HER2 inhibitor, and wherein a HER2-negative Status indicates the patient is unlikely to respond to treatment with a HER2 inhibitor.

In some embodiments, the HER2 inhibitor is trastuzumab.

In some embodiments, the tumor sample is obtained from the breast of the cancer patient.

### BRIEF DESCRIPTION OF THE FIGURES

FIG. 1 shows a graph showing HER2 distribution by QRT-PCR and IHC.

FIG. 2 shows a graph showing shows the 94% reduction in HER2-equivocal classifications using QRT-PCR vs. IHC.

FIG. 3 shows a graph of HER2 expression and ER expression by QRT-PCR.

FIG. 4 shows a graph of HER2 expression by Central FISH and QRT-PCR.

The present invention is not to be limited in scope by the specific embodiments or examples described herein. Modifications of the invention in addition to those described herein will be apparent to those skilled in the art from the foregoing description and the accompanying figures. Such modifications are intended to fall within the scope of this application. It is further understood that all values are approximate, and are provided for description. Patents, patent applications, publications, product descriptions, and protocols are cited throughout this application, the disclosures of which are hereby incorporated by reference in their entireties for all purposes.

### DETAILED DESCRIPTION

### Definitions

The terms “subject”, “individual”, and “patient” are used interchangeably herein to refer to a mammal being assessed for treatment and/or being treated. In an embodiment, the mammal is a human. The terms “subject”, “individual”, and “patient” thus encompass individuals having cancer (e.g., breast cancer) as well as individuals that do not have cancer, including those who have undergone or are candidates for resection (surgery) to remove cancerous tissue (e.g., cancerous breast tissue).

The terms “gene product” and “expression product” are used interchangeably herein in reference to a gene (e.g. HER2) to refer to the RNA transcription products (transcripts) of the gene, including mRNA and the polypeptide translation products of such RNA transcripts, whether such product is modified post-translationally or not. The terms “gene product” and “expression product” are used interchangeably herein, in reference to RNA, particularly an mRNA, to refer to the polypeptide translation products of such RNA, whether such product is modified post-translationally or not.

The term “cancer” or “tumor,” as used herein, means the physiological condition in mammals that is typically characterized by unregulated cell growth. Examples of cancer include but are not limited to, carcinoma, lymphoma, blastoma, sarcoma, melanoma, and leukemia. More particular examples of such cancers include squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, and various types of head and neck cancer.

The term “tissue sample” or “sample,” as used herein, refers to a tissue sample is meant a single part or piece of a tissue sample, e.g., a thin slice of tissue or cells cut from a tissue sample. It is understood that multiple sections of tissue samples may be taken and subjected to analysis, and the same section of tissue sample may be analyzed at both morphological and molecular levels, or may be analyzed with respect to both protein and nucleic acid.

The terms “ERBB2” and “HER2,” as used herein, interchangeably to refer to native sequence human HER2 protein described, for example, in Semba et al., (Proc. Natl. Acad. Sci USA 82:6497-6501 (1985)) and Yamamoto et al. (Nature 319:230-234 (1986)) (Genebank accession number X03363), and variants thereof. The term erbB2 refers to the gene encoding human HER2 and neu refers to the gene encoding rat p185neu. Preferred HER2 is native sequence human HER2. Examples of antibodies which bind HER2 include MAbs 4D5 (ATCC CRL 10463), 2C4 (ATCC HB-12697), 7F3 (ATCC HB-12216), and 7C2 (ATCC HB-12215) (see, U.S. Pat. No. 5,772,997; PCT Publication No. WO 98/17797; and U.S. Pat. No. 5,840,525, expressly incorporated herein by reference). Humanized anti-HER2 antibodies include huMAb4D5-1, huMAb4D5-2, huMAb4D5-3, huMAb4D5-4, huMAb4D5-5, huMAb4D5-6, huMAb4D5-7, and huMAb4D5-8 (HERCEPTIN™) as described in Table 3 of U.S. Pat. No. 5,821,337, which is expressly incorporated herein by reference; and humanized 520C9 (PCT Publication No. WO 93/21319). Human anti-HER2 antibodies are described in U.S. Pat. No. 5,772,997 and PCT Publication No. WO 97/00271.

The term “HER2 Status” as used herein refers to the classification of a tumor as HER2-Postitive, HER2-Negative or HER2-Equivocal. HER2 Status may also be used in reference to a tumor tissue sample or the patient from whom the tumor is obtained.

“HER2-Positive” as used herein refers to a sample from a tissue, or the patient or tumor from the tissue is obtained, that is classified as having a level of HER2 that is higher than the level of HER2 in a non-cancerous tissue of the same origin. The classification may result from measurement of either HER2 copy number (e.g. by ISH) or from measurement of the level of a HER2 expression product (e.g. by IHC or QRT-PCR). Classification of a tumor as HER2-Positive identifies the tumor as more likely to respond to HER2 inhibitors such as trastuzumab.

“HER2-Negative” as used herein refers to a sample from a tissue, or the patient or tumor from the tissue is obtained, that is classified as having a level of HER2 that is the same as or lower than the level of HER2 in a non-cancerous tissue of the same origin. The classification may result from measurement of either HER2 copy number (e.g. by ISH) or from measurement of the level of a HER2 expression product (e.g. by IHC or QRT-PCR). Classification of a tumor as HER2-Negative identifies the tumor as unlikely to respond to HER2 inhibitors such as trastuzumab.

The terms “HER2 cutpoint” and “HER2 expression cutpoint,” as used herein, mean a quantitative measure of HER2 expression which defines a boundary between tumors of different HER2 Status, e.g. between HER2-Negative tumors and HER2-Equivocal tumors (herein referred to as “lower HER2 expression cutpoint”) or between HER2-Equivocal tumors and HER2-Positive tumors (herein referred to as “upper HER2 expression cutpoint”). The terms “cutpoint”, “threshold” and “cut-off” may be used interchangeably in certain cases.

The term “ER Status” as used herein refers to the classification of a tumor as estrogen receptor (ER)-Positive and ER-Negative. ER Status may also be used in reference to a tumor tissue sample or the patient from whom the tumor is obtained.

“ER-Positive” as used herein refers to a sample from a tissue, or the patient or tumor from which the tissue is obtained, that contains cells that have a receptor protein (>10%) that binds the hormone estrogen. Cancer cells that are ER+ may need estrogen to grow, and may stop growing or die when treated with substances that block the binding and actions of estrogen. ER+ breast cancer tumors are generally sensitive to hormonal therapy, such as tamoxifen.

“ER-Negative” as used herein refers to a sample from a tissue, or the patient or tumor from the tissue is obtained, that contains cells that do not have a receptor protein (<10%) to which the hormone estrogen will bind. Cancer cells that are ER− do not need estrogen to grow. Classification of a tumor as ER-Negative identifies the tumor as unlikely to respond to hormonal therapy, such as trastuzumab. Xx

The terms “ER cutpoint” and “ER expression cutpoint,” as used herein, mean a quantitative measure of ER expression which defines a boundary between ER-Negative and ER-Positive tumors. The terms “cutpoint”, “threshold” and “cut-off” may be used interchangeably in certain cases.

“Concordance Rate,” as used herein, means that the overall accuracy of an assay combines sensitivity and specificity into a single measure of the percentage of cases (positive and negative, excluding equivocal cases) for which the assay result in concordant with the true status as measured by another validated test, such as IHC.

The term “validated test,” as used herein, refers to a diagnostic assay that has a demonstrated correlation between the HER2 Status determination with response to a breast or other cancer therapy. For example, both IHC and FISH are considered validated tests by the College of American Pathologists.

The term “pre-determined”, as used herein, means known beforehand. For example, in an assay that includes comparing an expression level to a pre-determined cutpoint, the cutpoint will be known before the assay is initiated.

The term “treatment” or “therapy,” as used herein, refers to both therapeutic treatment and prophylactic or preventative measures. Those in need of treatment include those already with the disorder as well as those in which the disorder is to be prevented.

The terms “chemotherapy” or “chemotherapeutic agent,” as used herein, refers to a chemical compound useful in the treatment of cancer.

As used herein, the term “normalized expression level” refers to an expression level of a test gene relative to the level of an expression product of one or more reference genes.

As used herein, the term “IHC” or “immunohistochemistry” refers to the process of localizing proteins in cells of a tissue section. This method employs antibodies that bind to specific antigens in biological tissues. IHC may be used to understand the distribution and localization of biomakers and differentially expressed proteins.

As used herein, the term “FISH” or “fluorenscence in situ hybridization” refers to a cytogenetic technique used to detect and localize the presence or absence of specific nucleic acid sequences. FISH uses fluorescent probes that bind target sequences to define patterns of gene expression within cells and tissues.

### DETAILED DESCRIPTION

Certain aspects of this disclosure related to a method for determining the HER2 status of a sample (e.g., a sample of a cancerous tissue) by: a) measuring the HER2 expression level from a sample using quantitative reverse transcriptase polymerase chain reaction (QRT-PCR); b) normalizing the HER2 expression level relative to at least one reference gene to generate a normalized HER2 expression level, c) comparing the normalized HER2 expression level to predetermined HER2 expression cutpoints to determine a HER2 status. In certain embodiments, the predetermined HER2 expression cutpoints are validated in that the HER2 expression level for a number of reference samples (which may be HER-positive or HER-negative) is evaluated beforehand by both QRT-PCR and at least one validated test (e.g., IHC and/or FISH). This data is used to calculate the cut-offs (the “cutpoints”) by which HER-positive samples and HER− negative samples can be accurately distinguished, e.g., with a confidence of 95% or greater. In general terms, the method may result in one of three outcomes: a) HER− positive, b) HER-negative and c) equivocal (i.e., uncertain). While the exact numerical values of the cutpoints employed in an assay may vary based on the number and type of reference samples evaluated to generate the cutpoints, the use of both QRT-PCR and the cutpoints in the subject method provides a highly accurate determination of whether a sample is HER2-positive or HER2-negative, and a low number of equivocal results as compared to other methods. In certain embodiments, less then 17% (e.g., less then 8%, less then 6%, less than 5%, less then 4%, less then 2%, less then 1% or less then 0.5%) of the test samples will be equivocal.

In certain embodiments, a different HER2 expression cutpoint is used depending on the ER status of the tissue. This allows accurate identification of three biologically distinct sub-groups of breast cancer patients: HER2/ER-negative (sometimes referred to as basal group), HER2-negative/ER-positive (sometimes referred to as luminal group), and HER2-positive. The risk profiles and recommended treatments for these three sub-groups are different. Thus, the method provides an accurate basis for selecting patients for treatment with appropriate chemotherapeutic agents, e.g., hormone receptor inhibitors. Certain embodiments of the subject method is believed to be more accurate than other IHC and FISH methods because: a) the number of equivocal samples obtained using the subject method is significantly lower relative to IHC and FISH-based methods and b) the majority of the samples that are deemed equivocal using another method (e.g., IHC or FISH) can be unequivocally designated as being either HER2-positive or HER2-negative using the subject method.

### Clinical Utility

The methods described herein will allow clinicians to better identify which patients will respond to hormonal therapy, and which tumors should be treated with other chemotherapeutic therapies. Breast cancer tumors that are HER2-positive are more aggressive and more likely to recur than HER2-negative tumors. Overactivity of HER receptors plays a role in the growth of many cancers and in their resistance to cancer therapy. For these reasons, HER2-positive cancers are considered high risk. Trastuzumab is a monoclonal antibody that works by binding the HER2 receptor. Studies have demonstrated that women with HER2-positive breast cancer treated with trastuzumab and chemotherapy lived longer and had significantly less chance of recurrence than those who were treated with chemotherapy alone. Lapatinib is a small molecule that acts on a number of proteins, including the HER2 receptor. Lapatinib is approved for use in combination with the chemotherapy drug capecitabine. Both of these therapies are only effective in HER2-positive breast cancer.

Estrogen is known to play an important role in breast cancer and about 70% of all such tumors are ER-positive. Hormonal therapies, such as tamoxifen, function in ER-positive breast cancer by blocking the estrogen receptor.

The efficacy of various treatments is significantly impacted by the HER2 and ER status of a tumor. According to NCCN Guidelines, patients that are (1) ER-positive should be treated with hormonal therapy, and (a) if HER2-positive, with chemotherapy and a HER2-inhibitor (e.g., trastuzumab); or (b) if HER2-negative, with chemotherapy; and (2) ER-negative and (a) HER2-positive should be treated with chemotherapy and a HER2-inhibitor; or (b) HER2-negative should be treated with chemotherapy. (See NCCN Guidelines v.1.2009, available online at www.nccn.org/professionals/physicians_gls/PDF/breast.pdf.)

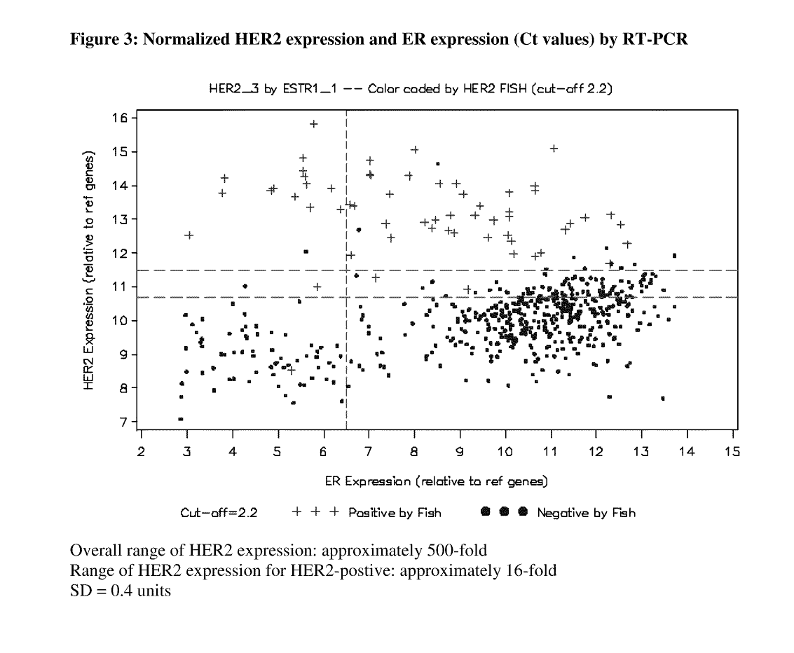

Thus, this method provides an accurate means to identify these various sub-groups of breast cancer tumors to ensure that patients are treated accurately based on their specific molecular profile. In some embodiments, the patients that would normally be classified as HER2-equivocal are re-classified as either HER2-positive or HER2-negative using HER2 expression cutpoints that vary depending on their ER status. FIG. 3 shows a graph of tumors categorized by HER2 Status and ER Status by QRT-PCR. Using this figure as an example, the HER2 expression cutpoint for tumors that are ER-negative would be the upper HER2 expression cutpoint (e.g., 11.5 Cₜ), and the HER2 expression cutpoint for ER-positive tumors would be the lower HER2 expression cutpoint (e.g., 10.7 Cₜ).

### Sample Preparation and QRT-PCR

The first step is the isolation of mRNA from a target sample. The starting material may be total RNA isolated from a human tumor or tumor cell line. Thus RNA can be isolated from a variety of primary tumors, including breast, lung, colon, prostate, brain, liver, kidney, pancreas, spleen, thymus, testis, ovary, uterus, head and neck, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

A target mRNA can be amplified by reverse transcribing the mRNA into cDNA, and then performing PCR (reverse transcription-PCR or RT-PCR). Alternatively, a single enzyme may be used for both steps as described in U.S. Pat. No. 5,322,770.

Once obtained, the level of HER2 mRNA in the sample may be determined using any quantitative method. In certain embodiments, a quantitative RT-PCR (“QRT-PCR”) method may be employed. In certain embodiments, the QRT-PCR method may be a so-called “real-time” RT-PCR method, although other conventional (e.g., gel-based) methods may be employed. Three exemplary real-time detection methodologies exist: (i) increased fluorescence of double strand DNA specific dye binding, (ii) decreased quenching of fluorescence during amplification, and (iii) increased fluorescence energy transfer during amplification. All of these techniques are non-gel based and each strategy is disclosed.

A variety of dyes are known to exhibit increased fluorescence in response to binding double stranded DNA. Production of wild type or mutation containing PCR products are continuously monitored by the increased fluorescence of dyes such as ethidium bromide or Syber Green as they bind to the accumulating PCR product.

A second detection technology for real-time PCR, known generally as exonuclease primers (e.g., TaqMan® probes), utilizes the 5′ exonuclease activity of thermostable polymerases such as Taq to cleave dual-labeled probes present in the amplification reaction (Wittwer, C. et al. Biotechniques 22:130-138, 1997; Holland, P et al PNAS 88:7276-7280, 1991, incorporated herein by reference). While complementary to the PCR product, the probes used in this assay are distinct from the PCR primer and are dually-labeled with both a molecule capable of fluorescence and a molecule capable of quenching fluorescence. When the probes are intact, intramolecular quenching of the fluorescent signal within the DNA probe leads to little signal. When the fluorescent molecule is liberated by the exonuclease activity of the polymerase during amplification, the quenching is greatly reduced leading to increased fluorescent signal. Taqman is a registered trademark of Roche Molecular Systems, Inc.

An additional form of real-time PCR also capitalizes on the intramolecular quenching of a fluorescent molecule by use of a tethered quenching moiety. The molecular beacon technology utilizes hairpin-shaped molecules with an internally-quenched fluorophore whose fluorescence is restored by binding to a DNA target of interest (Kramer, R. et al. Nat. Biotechnol. 14:303-308, 1996, incorporated herein by reference).

A final, general fluorescent detection strategy used for the measurement of the abundance of PCR products in real time utilizes synthetic DNA segments known as hybridization probes in conjunction with a process known as fluorescence resonance energy transfer (FRET) (Wittwer, C. et al. Biotechniques 22:130-138, 1997; Bernard, P. et al. Am. J. Pathol. 153:1055-1061, 1998, incorporated herein by reference). This technique relies on the independent binding of labeled DNA probes on the target sequence. The close approximation of the two probes on the target sequence increases resonance energy transfer from one probe to the other, leading to a unique fluorescence signal.

In one exemplary embodiment, the fluorogenic 5′ nuclease assay, known as the TAQMAN® assay (Perkin-Elmer), may be employed. For a detailed description of the TAQMAN assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties. Hence, primers and probes derived from regions of a target nucleic acid as described herein can be used in TAQMAN® analyses to detect a level of target mRNA in a biological sample. Analysis is performed in conjunction with thermal cycling by monitoring the generation of fluorescence signals.

The fluorogenic 5′ nuclease TAQMAN assay may be performed using, for example, AMPLITAQ GOLD® DNA polymerase, which has endogenous 5′ nuclease activity, to digest an internal oligonucleotide probe labeled with both a fluorescent reporter dye and a quencher (see, Holland et al., Proc. Natl. Acad. Sci. USA (1991) 88:7276-7280; and Lee et al., Nucl. Acids Res. (1993) 21:3761-3766). Assay results are detected by measuring changes in fluorescence that occur during the amplification cycle as the fluorescent probe is digested, uncoupling the dye and quencher labels and causing an increase in the fluorescent signal that is proportional to the amplification of target nucleic acid. Amplitaq Gold is a registered trademark of Roche Molecular Systems, Inc.

The amplification products can be detected in solution or using solid supports. In this method, the TAQMAN probe is designed to hybridize to a target sequence within the desired PCR product. The 5′ end of the TAQMAN probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. Excision of the 5′ fluorophore results in an increase in fluorescence which can be detected.

TaqMan RT-PCR can be performed using commercially available equipment, such as, for example, ABI PRISM® 7700 Sequence Detection System (Perkin-Elmer-Applied Biosystems, Foster City, Calif., USA), or Lightcycler® (Roche Molecular Biochemicals, Mannheim, Germany). In a preferred embodiment, the 5′ nuclease procedure is run on a real-time quantitative PCR device such as the ABI PRISM® 7700 Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

Factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. can be used.

### Normalization

To minimize the effect of sample-to-sample variation, quantitative RT-PCR is usually performed using an internal standard, or one or more reference genes. The ideal internal standard is expressed at a constant level among different tissues, and is unaffected by the experimental treatment. RNAs that can be used to normalize patterns of gene expression include, e.g., mRNAs for the reference genes glyceraldehyde-3-phosphate-dehydrogenase (GAPDH) and β-actin.

In carrying out a subject method, a level of HER2 mRNA in a sample from a patient is assayed by a quantitative method, as described above. The level HER2 mRNA is then “normalized” relative to the level of expression of the mRNA of one of more reference genes, thereby generating a normalized expression level of HER2 mRNA.

For example, the level of HER2 mRNA can be normalized relative to the mean level of gene products of two or more reference genes. As an example, the level of HER2 mRNA can be normalized relative to the mean level of gene products of all assayed genes, or a subset of the assayed genes, where a subset of the assayed genes can include 3, 4, 5, 6, 7, 8, 9, or more assayed genes. As one non-limiting example, the expression level of a response indicator gene can be normalized to the mean expression level of the following reference genes: ACTB, GAPDH, GUSB, RPLP0 and TFRC. Those skilled in the art will readily appreciate that other combinations of genes can be used as reference genes for the purposes of determining a normalized level HER2 mRNA. Additional suitable reference genes include, but are not limited to, glyceraldehyde-3-phosphate dehydrogenase (GAPDH) (see, e.g., GenBank Accession No. NM_{—}002046; phosphoglycerate kinase 1 (see, e.g., GenBank Accession No. NM_{—}000291); lactate dehydrogenase A (see, e.g., GenBank Accession No. NM_{—}005566); ribosomal protein L32 (see, e.g., GenBank Accession No. NM_{—}000994); ribosomal protein S18 (see, e.g., GenBank Accession No. NM_{—}022551); tubulin, beta polypeptide (TUBB) (see, e.g., GenBank Accession No. NM_{—}001069); and beta actin (see, e.g., GenBank Accession No. NM_{—}001101). See, e.g., Eisenberg and Levanon (2003) Trends in Genetics 19:362, for a list of additional suitable reference genes.

The level of an RNA transcript as measured by TaqMan® RT-PCR refers to the cycle threshold (Ct) value. The lower the Cₜ, the greater the amount of mRNA present in the sample. The expression value of a RNA transcript in a sample is normalized, e.g., by first determining the mean expression value in Cₜ of designated reference genes in a is sample (Ct_{Ref}). The normalized expression value for a gene (Ct_{Gene}) is then calculated as Ct_{Gene}-Ct Ct_{Ref}. Optionally, the normalized expression values for all genes can be adjusted, e.g., so that all adjusted normalized Cₜ have a value >0.

### Comparison to Cutpoints

After a normalized HER2 expression level is obtained, it is compared to predetermined HER2 expression cutpoints to determine a HER2 status. In certain embodiments, the cutpoints define the numerical boundaries between a) normalized expression levels that are HER2-positive and equivocal expression levels (i.e., the “upper” cutpoint) and b) normalized expression levels that are HER2-negative and equivocal expression levels (i.e., the “lower” cutpoint). If a normalized HER2 expression level is not equivocal, the normalized HER2 expression level can be unequivocally designated as either a HER2-positive or HER2-negative expression level. Thus, the sample from which the normalized HER2 expression level was obtained can be designated as a HER2-positive or HER2-negative sample if it is not equivocal.

The HER2 expression cut-points may vary depending on the ER Status of the tumor. The HER2 expression cut-point for an ER+ tumor would be the upper HER2 expression cutpoint. The HER2 expression cut-point for an ER− tumor would be the lower HER2 expression cutpoint.

As noted above, the cutpoints are statistically validated in that they have been “trained” on prior samples that are known (via other, “validated” methods, e.g., FISH or IHC) to be either HER2-positive or HER2-negative. Given a set of training samples that are of known HER2 status, alternatively defined cutpoints for assays based on QRT-PCR or other technologies test assays can be readily determined using a variety of statistical tests that are known in the art. Such statistical tests may include, but are not limited to: Pearson's Correlation, T-test, Mann-Whitney U test, binomial test, Wilcoxon signed-rank test, analysis of variance, as well as many others.

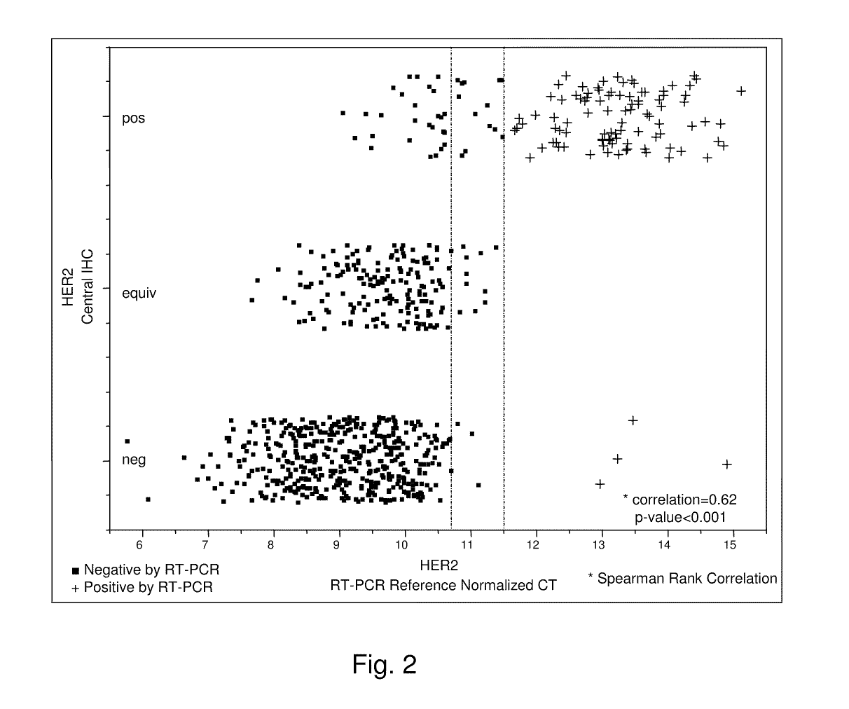

Once the cutpoints are determined using training samples, it is important that a test sample is assayed using the same assay parameters (e.g., the same primers, amplification conditions, normalization controls, data processing methods, etc.) as the training samples so that the results obtained using the test sample can be directly compared to the cutpoints to determine the HER2 status of the test sample. For example, using the method described in the examples section of this disclosure and with reference to FIG. 2, a test sample that had a normalized Cₜ of less than 10.7 would be unequivocally HER2-negative, whereas a test sample that had a normalized Cₜ of greater than or equal to 11.5 would be unequivocally HER2-positive. Those test samples that have a normalized Cₜ greater than or equal to 10.7 and less than 11.5 would be equivocal.

### Analysis Results Reporting

The method described above may be employed to determine the likelihood that a patient will exhibit a beneficial response to an HER2 inhibitor treatment. In some embodiments, a patient's likelihood of response to HER2 inhibitor treatment is provided in a report. Thus, in some embodiments, a subject method further includes a step of preparing or generating a report that includes information regarding the patient's likelihood of response. For example, a subject method can further include a step of generating or outputting a report providing the results of a subject response likelihood assessment, which report can be provided in the form of an electronic medium (e.g., an electronic display on a computer monitor), or in the form of a tangible medium (e.g., a report printed on paper or other tangible medium).

A report that includes information regarding the likelihood that a patient will respond to an HER2 inhibitor treatment is provided to a user. An assessment as to the likelihood that a patient having an HER2-expressing cancer will respond to treatment with an HER2 inhibitor is referred to below as a “response likelihood assessment” or, simply, “likelihood assessment.” A person or entity who prepares a report (“report generator”) will also perform the likelihood assessment. The report generator may also perform one or more of sample gathering, sample processing, and data generation, e.g., the report generator may also perform one or more of: a) obtaining a sample (e.g., receiving a sample); b) sample processing; c) measuring a level of HER2 gene product(s); d) measuring a level of a reference gene product(s); and e) determining a normalized level of HER2 gene product(s) and f) comparing the normalized level to the cutoffs to determine if the sample is HER2 positive or negative. Alternatively, an entity other than the report generator can perform one or more sample gathering, sample processing, and data generation.

For clarity, it should be noted that the term “user,” which is used interchangeably with “client,” is meant to refer to a person or entity to whom a report is transmitted, and may be the same person or entity who does one or more of the following: a) collects a sample; b) processes a sample; c) provides a sample or a processed sample; and d) generates data (e.g., level of a response indicator gene product(s); level of a reference gene product(s); normalized level of a response indicator gene product(s)) for use in the likelihood assessment. In some cases, the person(s) or entity(ies) who provides sample collection and/or sample processing and/or data generation, and the person who receives the results and/or report may be different persons, but are both referred to as “users” or “clients” herein to avoid confusion. In certain embodiments, e.g., where the methods are completely executed on a single computer, the user or client provides for data input and review of data output. A “user” can be a health professional (e.g., a clinician, a laboratory technician, a physician (e.g., an oncologist), etc.).

In embodiments where the user only executes a portion of the method, the individual who, after computerized data processing according to certain of the methods described herein, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

A “report,” as described herein, is an electronic or tangible document which includes report elements that provide information of interest relating to a subject likelihood assessment and its results. A subject report includes at least a likelihood assessment, e.g., an indication as to the likelihood that a patient having an EGFR-expressing cancer will exhibit a beneficial clinical response to an EGFR inhibitor treatment regimen. A subject report can be completely or partially electronically generated. A subject report can further include one or more of: 1) information regarding the testing facility; 2) service provider information; 3) patient data; 4) sample data; 5) an interpretive report, which can include various information including: a) indication; b) test data, where test data can include: i) a normalized level of HER2 and ii) cutpoints; and 6) other features.

Where government regulations or other restrictions apply (e.g., requirements by health, malpractice, or liability insurance), all results, whether generated wholly or partially electronically, are subjected to a quality control routine prior to release to the user.

The report can include information about the testing facility, which information is relevant to the hospital, clinic, or laboratory in which sample gathering and/or data generation was conducted. Sample gathering can include obtaining a cancer cell sample from a biopsy, a surgically removed tumor, surgically removed tissue comprising a tumor, or other tissue or bodily fluid from a patient. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report can include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The patient data can include patient medical history (which can include, e.g., data about prior treatment for cancer), personal history; administrative patient data (that is, data that are not essential to the likelihood assessment), such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the response likelihood assessment and, if different from the ordering physician, the name of a staff physician who is responsible for the patient's care (e.g., primary care physician). Report fields with this information can generally be populated using data entered by the user.

The sample data can provide information about the biological sample analyzed in the likelihood assessment, such as the source of biological sample obtained from the patient (e.g., tumor biopsy, surgically removed tumor, unknown, etc.) and the date and time collected. Report fields with this information can generally be populated using data entered by the user, some of which may be provided as pre-scripted selections (e.g., using a drop-down menu).

The interpretive report portion of the report includes information generated after processing of the data as described herein. The interpretive report can include an indication of the likelihood that the patient will respond to treatment with an HER2 inhibitor. The interpretive report can include, for example, Interpretation; and, optionally, Recommendation(s).

The Interpretation portion of the report can include a Recommendation(s). Where the results indicate a likelihood of beneficial response to an HER2 inhibitor treatment, the recommendation can include a recommendation that an HER2 inhibitor regimen is indicated. Where the results indicate that a beneficial response to an HER2 inhibitor treatment is not likely, the recommendation can include a recommendation for an alternative treatment regimen.

It will be readily appreciated that the report can include all or some of the elements above, with the proviso that the report generally includes at least the elements sufficient to provide the analysis requested by the user (e.g., likelihood assessment).

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting.

### Computer-Based Systems and Methods

The methods described herein can be implemented in numerous ways. In one embodiment of particular interest, the methods involve use of a communications infrastructure, for example the internet. Several embodiments of the invention are discussed below. It is also to be understood that the present invention may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site associated (e.g., at a service provider's facility).

For example, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which can include test data (e.g., level of a HER2 gene product(s); level of a reference gene product(s); normalized level of a response indicator gene product(s)); and may also include other data such as patient data. This information received can be stored at least temporarily in a database, and data analyzed to generate a report as described above.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In an embodiment of particular interest, all or a portion of the input data and/or all or a portion of the output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record which may exist in a confidential database at the healthcare facility.

A system for use in the methods described herein generally includes at least one computer processor (e.g., where the method is carried out in its entirety at a single site) or at least two networked computer processors (e.g., where data is to be input by a user (also referred to herein as a “client”) and transmitted to a remote site to a second computer processor for analysis, where the first and second computer processors are connected by a network, e.g., via an intranet or internet). The system can also include a user component(s) for input; and a reviewer component(s) for review of data, generated reports, and manual intervention. Additional components of the system can include a server component(s); and a database(s) for storing data (e.g., as in a database of report elements, e.g., interpretive report elements, or a relational database (RDB) which can include data input by the user and data output. The computer processors can be processors that are typically found in personal desktop computers (e.g., IBM, Dell, Macintosh), portable computers, mainframes, minicomputers, or other computing devices.

The input client components can be complete, stand-alone personal computers offering a full range of power and features to run applications. The client component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

### Computer-Readable Storage Media

Also provided is a computer-readable storage medium (e.g. CD-ROM, memory key, flash memory card, diskette, etc.) having stored thereon a program which, when executed in a computing environment, provides for implementation of algorithms to carry out all or a portion of the analysis (e.g., the normalization and/or comparison steps) as described herein. Where the computer-readable medium contains a complete program for carrying out the analysis, the program includes program instructions for collecting, analyzing and generating output, and generally includes computer readable code devices for interacting with a user as described herein, processing that data in conjunction with analytical information, and generating unique printed or electronic media for that user.

Where the storage medium provides a program which provides for implementation of a portion of the methods described herein (e.g., the user-side aspect of the methods (e.g., data input, report receipt capabilities, etc.)), the program provides for transmission of data input by the user (e.g., via the internet, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out response likelihood assessment (e.g., primers, probes, arrays, or other such kit components).

### Method of Treatment

If a sample is designated a HER2-positive sample, then the subject from which the sample was obtained may be subjected to a treatment regimen that includes a HER2 inhibitor such as a monoclonal antibody that specifically binds to and kills cells expressing HER2. Such antibodies include trastuzumab (sold under the trade name Herceptin®) which is a recombinant humanized anti-HER2 monoclonal antibody used for the treatment of HER2 over-expressed/HER2 gene amplified metastatic breast cancer. Herceptin is a registered trademark of Genentech, Inc. Trastuzumab binds specifically to the same epitope of HER2 as the murine anti-HER2 antibody 4D5 described in Hudziak, et al., Mol. Cell. Biol. 9 (1989) 1165-1172. Trastuzumab is a recombinant humanized version of the murine anti-HER2 antibody 4D5, referred to as rhuMAb 4D5 or trastuzumab) and has been clinically active in patients with HER2-over expressing metastatic breast cancers that had received extensive prior anticancer therapy. (Baselga, et al, J. Clin. Oncol. 14 (1996) 737-744). Trastuzumab and its method of preparation are described in U.S. Pat. No. 5,821,337. Pertuzumab (Omnitarg™, Genentech, Inc.) is another recombinant humanized anti-HER2 monoclonal antibody used for the treatment of HER2 positive cancers. Pertuzumab binds specifically to the 2C4 epitope, a different epitope on the extracellular domain of HER2 as trastuzumab. Pertuzumab is the first in a new class of HER dimerisation inhibitors (HDIs). Through its binding to the HER2 extracellular domain, pertuzumab blocks ligand-activated heterodimerisation of HER2 with other HER family members, thereby inhibiting downstream signalling pathways and cellular processes associated with tumour growth and progression (Franklin, M. C., et al. Cancer Cell 5 (2004) 317-328 and Friess, T, et al. Clin Cancer Res 11 (2005) 5300-5309). Pertuzumab is a recombinant humanized version of the murine anti-HER2 antibody 2C4 (referred to as rhuMAb 2C4 or pertuzumab) and it is described together with the respective method of preparation in WO 01/00245 and WO 2006/007398. Such an antibody may be administered alone or in conjunction with other chemotherapeutic agent such as IL-2.

A variety of publications describe anti-HER2 monoclonal antibodies and treatment regimens using the same including, for example, published patent applications 20080159981, 20080102069, 20070142346, 20060018899, 20050148607 and 20030228663, as well as U.S. Pat. Nos. 7,306,801, 6,399,063, 6,387,371 and 6,165,464, which are each incorporated by reference in their entirety. As such, these antibodies and methods need not be described herein in any great detail. In general terms, these documents described methods including administering a therapeutically anti-HER2 monoclonal antibody to a HER2-positive subject, singly or in combination with another chemotherapeutic agent. The subject may then be monitored for a clinically beneficial response, where a beneficial response to the antibody can be assessed according to whether an individual patient experiences a desirable change in disease status. Examples of desirable changes in disease status in cancer include loss of detectable tumor (complete response, (CR), decrease in tumor size and/or cancer cell number (partial response, PR), and tumor growth arrest (stable disease, SD)). Continued increase in tumor size and/or cancer cell number and/or tumor metastasis is indicative of lack of beneficial response to treatment.

### Kits

Also provided herein are kits for practicing the subject method, as described above. The subject kit may contain sequence-specific primers for performing quantitative RT-PCR analysis of HER2 mRNA in a sample (or probes for performing quantitative analysis of HER2 mRNA in a sample using other methods). The kit may further contain a reverse transcriptase, reagents for real-time PCR (e.g., a buffer, nucleotides, etc), materials for fluorescent labeling of polymerase products, a reference sample to be employed in the subject method.

In additional embodiments, the kit further comprises sequence-specific primers for performing quantitative RT-PCR analysis (or other quantitative analysis) of reference mRNAs from the sample. Results obtained using the HER2 primers may be normalized using results obtained from the primers for the reference genes.

In addition to above-mentioned components, the subject kit may further include instructions for using the components of the kit to practice the subject methods, e.g., instructions on how to perform the quantitative analyses and normalize results. The kit may further contain the cutpoints to which the normalized HER2 level may be compared to determine whether the sample is a HER2-positive or HER2-negative sample. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The various components of the kit may be in separate containers.

### HER2 Status in Breast Cancer

Among breast cancer patients, the status of human epidermal growth factor receptor-2 (HER-2) is an important prognostic marker that guides medical treatment decisions. The tyrosine kinase signaling network includes a group of HER receptors that are associated with the development of malignant tumors. The overexpression of, or failure to block signals of the HER receptors, including HER2, leads to uncontrollable growth. In the case of HER2, overexpression may be the result of a genetic alternation that generates multiple copies of a gene that encodes a growth receptor. Because of the surplus of growth receptor genes in the cell, excessive numbers of growth receptors are created that, when activated, increase the number of signals stimulating the cell, thereby accelerating cell division and tumor growth.

HER-2 positive breast cancer, which occurs in approximately 25% of women with breast cancer, is characterized by aggressive growth and a poorer prognosis. (Selvarajan; D. Slamon, et al., Use of the anti HER-2/neu antibody Herceptin in the treatment of human breast cancer: biological rationale and clinical results, Breast Cancer Res., 2 (Supp. 1):S.14 (March 2000).) Treatment decisions are based on HER2 status because breast cancers that are HER2-positive tend to shrink or stop growing when treated with therapies targeted to inhibit the HER2 pathway. For example, adjuvant anthracyclin-based treatment has been shown to have added benefit for HER2-positive breast cancer patients. (A. Gennari, et al., HER2 status and efficacy of adjuvant anthracyclines in early breast cancer: a pooled analysis of randomized trials, J. Nat'l Cancer Inst., 100 (1):2-4 (January 2008).) In addition, trastuzumab is a therapeutic antibody approved for the adjuvant treatment of patients with HER2-positive breast cancer.

The technologies most commonly used to determine HER2 overexpression are fluorescence in situ hybridization (FISH) and immunohistochemistry (IHC). However, as many as 20% of locally performed HER2 assays are determined to be false positive when tested by central laboratories using FISH and IHC. (E. Perez, et al., HER2 testing by local, central, and reference laboratories in specimens from the north central cancer treatment group N9831 intergroup adjuvant trial, J. Clin. Oncol., 1, 24(19):3032-3038 (July 2006).)

Analysis of HER2 status by FISH requires a fluorescent microscope and an image capture system and must be conducted by highly trained professionals. Consequently, FISH can be expensive and, in addition, FISH may not detect overexpression of HER2 that is not associated with HER2 gene amplification.

HER2 IHC is a protein-based diagnostic used to identify women whose breast carcinomas as HER2 positive for protein overexpression if there is strong, circumferential, cytoplasmic membrane staining greater than 30% of carcinoma cells (3+). Equivocal cases (2+) either show less than 30% tumor cells staining or do not show strong circumferential cytoplasmic membrane staining. Carcinoma cells with weak staining (1+) or no staining (0) are considered negative for protein overexpression. These scores are based on a subjective microscopic evaluation of both the percentage of tumor cells staining with an anti-HER2 antibody and the relative intensity of the staining observed.

Due to the semi-quantitative nature of the IHC HER2 test, a large proportion of results are equivocal (2+) as to the HER2 Status of the tumor. In addition, HER2 Status of individuals as determined by current testing methods, particularly IHC, can show poor laboratory-to-laboratory agreement. For these reasons, the NCCN recommends that a FISH test should be conducted to confirm HER2 status if the IHC results are equivocal.

Also, IHC of formaldehyde-fixed, paraffin embedded tissue samples only demonstrated 50-80% sensitivity relative to frozen IHC samples. (J. Ross, J. Fletcher, The HER-2/neu oncogene in breast cancer, Stem Cells, Vol. 16, No. 6:413-428 (November 1998).) Thus, IHC can also lead to false negative results, excluding from treatment patients who might benefit from treatment. In addition, IHC has been shown to have a relatively high (50%) false positive (3+) rate. (L. Hammock, et al., Strong HER-2/neu protein overexpression by immunohistochemistry often does not predict oncogene amplification by fluorescence in situ hybridization, Human Pathology, Vol. 34, Iss. 10:1043-1047 (2003).)

Herceptest®, an IHC test for HER2, was approved by the United States Food and Drug Administration in 1998, and, in spite of the shortcomings of IHC methodologies discussed above, is still today the most popular methodology for HER2 testing. Herceptest is a registered trademark of Genentech, Inc.

Studies have correlated HER2 determination by IHC (IHC 3+) and FISH with response to certain breast cancer therapies, including trastuzumab and chemotherapy. (M. Hofmann, et al., Central HER2 IHC and FISH analysis in a trastuzumab (Herceptin) phase II monotherapy study, J. Clin. Path., Vol. 61, n. 1:89-94 (2008); A. Laurent, et al., Pathologic complete response to trastuzumab-based neoadjuvant therapy is related to the level of HER-2 amplification, Clin. Cancer Res., 13: 6404-6409 (November 2007); T. Petit, et al., Chemotherapy response of breast cancer depends on HER-2 status and anthracycline does intensity in the neoadjuvant setting, Clin. Cancer Res., Vol. 7:1577-1581 (June 2001).) For this reason, these HER2 testing methods are described as “validated tests.”

The College of American Pathologists (CAP) and the American Society of Clinical Oncology (ASCO) issued HER2 assessment guidelines calling for improved accuracy and reproducibility of HER2 testing in invasive breast cancer. These organizations estimate that approximately 20% of current HER2 testing may be inaccurate. Despite careful validated testing, their data did not clearly demonstrate the superiority of either ICH or in situ hybridization (ISH) as a predictor of benefit from anti-HER2 therapy. The organizations proposed assay involving “newly available types of brightfield ISH.” The guidelines were intended to improve the accuracy of HER2 testing. Towards that end, the guidelines recommend that, to perform HER2 testing, laboratories show 95% concordance with a validated test for positive and negative assay values.

Certain embodiments of the method described herein are based, in part, on the unexpected discovery that HER2 gene amplification as detected by QRT-PCR provides a more accurate basis for selecting patients for treatment because QRT-PCR HER2 Status significantly reduces equivocal results as compared to IHC.

General methods for measuring the levels of gene expression products using quantitative RT-PCR are fully described in a co-pending patent application, U.S. application Ser. No. 11/653,102, the contents of which are hereby incorporated by reference. (See, also, M. Cronin, et al., Clin. Chem., 53(6):1084-1091 (June 2007).)

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### IHC HER2 Status

IHC and QRT-PCR were performed using samples from Eastern Cooperative Oncology Group (ECOG) Study E2197. (L. Goldstein, et al., Prognostic utility of the 21-gene assay in hormone receptor positive operable breast cancer compared with classical clinicopathologic features, J. Clin. Oncol., 26(25) (2008). In that study, breast cancer patients with 0-3 positive nodes were treated with either anthracycline-cyclophosphamide or anthracycline-docetaxel (n=2885). Median follow-up was 76 months and no difference in Disease Free Survival was observed between the two arms.

In a cohort sampling study, tumor samples were selected representing 776 patients (179 with recurrence; 597 without recurrence) in the E2197 trial. The patients included in this study had similar characteristics to those not included, with the exception of menopausal status (p=0.03) and the proportion with 2-3 positive nodes (p=0.05). Of 776 total samples, 755 had evaluable HER2 by central IHC and by central QRT-PCR. HER2 Status by IHC was evaluated using HercepTest® (Dako Denmark A/S; Glostup, DK) according to manufacturer's instructions. Slides were stained and evaluated centrally by two pathologists blinded to outcome based on percent positive cells and staining intensity.

### FISH HER2 Status

FISH and QRT-PCR were performed using samples from a Kaiser Permanent matched case-control study conducted among 4,964 patients diagnosed with node-negative breast cancer. (L. Habel, et al., A population-based study of tumor gene expression and risk of breast cancer death among lymph node-negative patients, Breast Cancer Res., 8(3):R25 (2006).) Incidence density sampling was used to match up to 3 controls per case. Therefore, a patient could be both a control (up until the time of death from breast cancer) and a case. The total unique number of patients was 568.

HER2 Status by FISH was evaluated using Vysis PathVysion® HER-2/neu DNA Probe Kit (Vysis, Inc./Abbott Laboratories, Abbott Park, Ill.) according to the manufacturer's instructions. Tumor grading was conducted using the Nottingham combined histologic grading system on hematoxylin and eosin (H&E) stained tumor whole sections. (E. Rakha, Prognostic significance of Nottingham histologic grade in invasive breast carcinoma, J. Clin. Oncol., 26(19):3153-3158 (July 2008).) Results were scored by 3 pathologists counting as least 60 tumors cells, and reported as a ratio of HER2 to chromosome 17 signals. Pathologists used the ASCO/CAP HER2 cutpoints for amplification ratio (HER2-positive: >2.2, HER2-equivocal: 1.8-2.2, HER2-negative: <1.8).

### Quantitative RT-PCR

After RNA extraction and DNase I treatment, total RNA content was measured and the absence of DNA contamination was verified.

Gene-specific reverse transcription was performed, followed by quantitative PCR (TaqMan®) assay in 384-well plates using ABI Prism® 7900HT instruments (Applied Biosystems, Foster City, Calif.). The nucleotides used for QRT-PCR were CGGTGTGAGAAGTGCAGCAA (forward primer; SEQ ID NO:1), CCTCTCGCAAGTGCTCCAT (probe; SEQ ID NO:2) and CCAGACCATAGCACACTCGGGCAC (reverse primer; SEQ ID NO:3), which served to prime the reverse transcription reaction and as a PCR amplification primer.

Gene expression values were normalized relative to a set of five reference genes (ACTB, GAPDH, GUSB, RPLP0 and TFRC). Reference-normalized expression measurements ranged from 0 to 15, where each 1-unit increase reflects about a two-fold increase in RNA. HER2 categories were prespecified using reference-normalized values (HER2-positive: ≧11.5, HER2-equivocal: ≧10.7 to <11.5, HER2-negative: <10.7).

### Calculation of Normalized HER2 Expression Values

Normalized expression measurements were derived based upon the results of RT-PCR assays involving triplicate RT-PCR plate wells per gene. The first step in this procedure is the retrieval and calculation of a cycle threshold (CT) measurement for each well. Each RT-PCR plate well has associated with it a set of amplification and quality metrics used to determine the quality and validity of each Cₜ measurement. For each gene, the triplicate Cₜ measurements were aggregated into a weighted average Cₜ for the valid wells. Methods for evaluating the quality and validity of PCR measurements and for weighting the contribution of each replicate to an aggregated average (for example, to minimize the contribution of outliers) are known in the art.

Systematic differences in RT-PCR Cₜ measurements can result between different oligonucleotide lots due to inherent variations in oligonucleotide syntheses. Due to potential differences in oligonucleotide sets, calibration methods are used to adjust for systematic differences between new oligonucleotide sets versus the baseline oligonucleotide sets used for clinical validation studies. Calibration utilizes a Human Universal Reference Standard RNA to account for mean shifts in aggregate Cₜ measurement between the new oligonucleotide lot versus the mean baseline Cₜ measurements for the baseline oligonucleotide sets used in clinical validations studies. Data calibration methods used to adjust for potential differences in oligonucleotide sets resulting for example from lot-to-lot manufacturing variations in the oligonucleotides comprising the oligonucleotide set are known in the art.

The resulting calibrated measurements are then normalized relative to the average of a set of five reference genes (ACTB, GAPDH, GUSB, RPLP0 and TFRC). Normalization occurs by subtraction of the observed Cₜ measurement for HER2 from the average Cₜ measurement for the reference gene set and adding 10.

### Demonstration of Concordance of HER2 Status Classification by QRT-PCR with HER2 Status Classification by IHC and FISH

HER2 status by QRT-PCR is classified according to pre-specified HER2 cutpoints and may be based on reference-normalized values. The HER2 cutpoints may be calculated using a high concordance rate with at least one validated method. As discussed above, CAP/ASCO HER2 testing guidelines recommend that laboratories show 95% concordance with another validated test for positive and negative assay values.

For purposes of this example, the cutpoint ranges used were as follows: HER2-positive (≧11.5), HER2-equivocal (≧10.7 to <11.5), and HER2-negative (<10.7). The HER2 cutpoint of 11.5 was derived from concordance rates from three prior studies involving populations of patients consisting of those both positive and negative for HER2 as assessed by both IHC and FISH. (See, F. Esteva, et al., Prognostic role of a multigene reverse transcriptase-PCR assay in patients with node-negative breast cancer not receiving adjuvant systemic therapy, Clin. Cancer Res., 11(9):3315-9 (May, 2005); M. Cobleigh, et al., Tumor gene expression and prognosis in breast cancer patients with 10 or more positive lymph nodes, Clin. Cancer Res., 11(24 Pt 1):8623-31 (Dec. 15, 2005); L. Gianni, et al., Gene expression profiles in paraffin-embedded core biopsy tissue predict response to chemotherapy in women with locally advanced breast cancer, J. Clin. Oncol., 23(29):7265-77 (October, 2005).) The cutpoint of 10.7 was derived by optimizing the association between normalized HER2 expression and recurrence risk using nonlinear Cox Proportional Hazards regression modeling of recurrence risk as a function of normalized HER2 expression measurement in samples derived from the B-14 study conducted by the National Surgical Adjuvant Breast and Bowel Project. Selection of these samples is described in U.S. Pat. No. 7,056,675 and copending U.S. application Ser. No. 10/883,303

Table 1 compares HER2 status classification as determined by QRT-PCR with status classification as determined by FISH. Table 1 omits those tumors which were classified by QRT-PCR and/or FISH as HER2-equivocal.

TABLE 1

| | Central FISH+ | | Central FISH− | | Total |
|---|---|---|---|---|---|
| Positive (QRT-PCR) | 55 | (98%) | 11 | (3%) | 66 |
| Negative (QRT-PCR) | 1 | (2%) | 408 | (97%) | 409 |
| Total | 56 | | 419 | | 475 |

Of the total of 568 tumors studied, 93 were HER2-equivocal by QRT-PCR and/or central FISH. Of the remaining 475 tumors studied, 55 were classified as HER2-positive by both QRT-PCR and central FISH, and 408 were classified as HER2-negative by both methods. The concordance rate between the methods was 97% ((55+408)/475). Thus, in 97% of the tumors for which HER2 Status could be classified by FISH as positive or negative, classification by QRT-PCR resulted in the same HER2 Status as classification by FISH.

Table 2 compares HER2 status classification as determined by QRT-PCR with status classification as determined by central IHC. Table 2 omits those tumors which were classified by QRT-PCR and/or central IHC as HER2-equivocal.

TABLE 2

| | Positive (IHC) | | Negative (IHC) | | Total |
|---|---|---|---|---|---|
| Positive (QRT-PCR) | 94 | (78%) | 4 | (1%) | 98 |
| Negative (QRT-PCR) | 27 | (22%) | 439 | (99%) | 466 |
| Total | 121 | | 443 | | 564 |

Of the total of 755 tumors studied, 191 were HER2-equivocal by QRT-PCR and/or central IHC. Of the remaining 564 tumors studied, 94 were classified as HER2-positive by both QRT-PCR and central IHC, and 439 were classified as HER2-negative by both methods. The concordance rate between the methods was 95% ((94+439)/564). Thus, in 95% of the tumors for which HER2 Status could be classified by IHC as positive or negative, classification by QRT-PCR resulted in the same HER2 Status as classification by IHC.

### Reduction in her2-Equivocal Results Using HER2 Status Classification by QRT-PCR

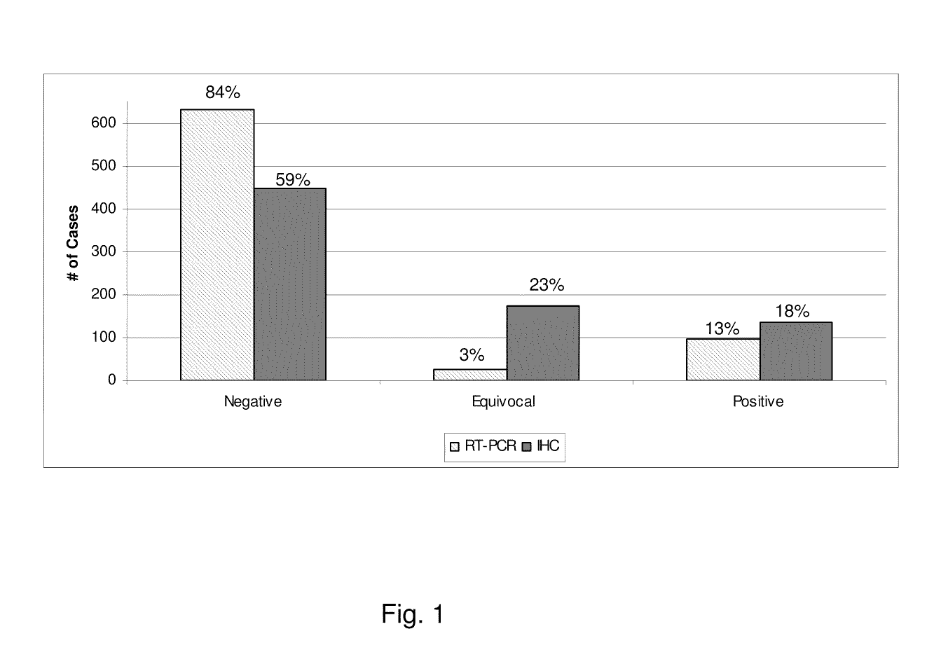

Of the 755 tumors studied, 23% were HER2-Equivocal by central IHC. Of the 175 tumors that were HER2-Equivocal by central IHC, 165 were classified by QRT-PCR, all of which were classified as HER2-Negative. FIG. 1 shows the HER2 distribution by QRT-PCR and IHC.

The quantitative nature and precision of QRT-PCR HER2 allowed the establishment of HER2 expression cutpoints for HER2 status classification, which led to a surprisingly substantial 94% reduction compared to HER2 status classification by IHC in the proportion of tumors for which the HER2 status was indeterminate. FIG. 2 shows the 94% reduction in HER2-equivocal classifications using QRT-PCR vs. IHC.

Table 3 compares HER2 status classification as determined by QRT-PCR with status classification as determined by central IHC. Table 3 includes all tumors including those which were classified by either or both QRT-PCR and central IHC as HER2-Equivocal. The same results are shown in graphical form in FIG. 2.

TABLE 3

| | Positive | | Equivocal | | Negative | | |
|---|---|---|---|---|---|---|---|
| | (IHC) | | (IHC) | | (IHC) | | Total |
| Positive | 94 | (70%) | 0 | | 4 | (<1%) | 98 |
| (QRT-PCR) | | | | | | | |
| Equivocal | 13 | (10%) | 10 | (6%) | 3 | (<1%) | 26 |
| (QRT-PCR) | | | | | | | |
| Negative | 27 | (20%) | 165 | (94%) | 439 | (99%) | 631 |
| (QRT-PCR) | | | | | | | |
| Total | 134 | | 175 | | 446 | | 755 |

### Statistical Analyses

2×2 tables of results comparing positive/negative by RT-PCR to positive/negative by FISH were computed. 3×3 tables of results comparing positive, equivocal, negative by RT-PCR to positive, equivocal, negative by FISH were computed.

Measures of agreement between RT-PCR and FISH include overall concordance, calculated as the number of samples that agree divided by the total number of samples and Kappa (or weighted Kappa) statistics (agreement adjusted for chance). Exact 95% confidence intervals for the concordance statistic were calculated with the F-distribution method using the PROC FREQ procedure in SAS V9. Percent positive agreement was calculated as the number of samples positive by both assays divided by the number of samples positive by the FISH assay. Conditional logistic regression was used to estimate the association between HER2 and risk of breast cancer death. All statistical tests were two-sided and p<0.05 was considered significant.

FIG. 3 demonstrates comparative HER2 and ER expression by QRT-PCR for tumors that were identified as HER2-positive (+) or HER2-negative (−) by FISH. The ER expression cutpoint is shown as a vertical dashed line (at 6.5 Cₜ), i.e., those samples to the left of this line are ER-negative and on or to the right, ER-positive. The HER2 expression cutpoints are shown as horizontal dashed lines. The lower HER2 expression cutpoint is shown at 10.7 Cₜ, and the upper HER2 expression cutpoint is shown at 11.7.

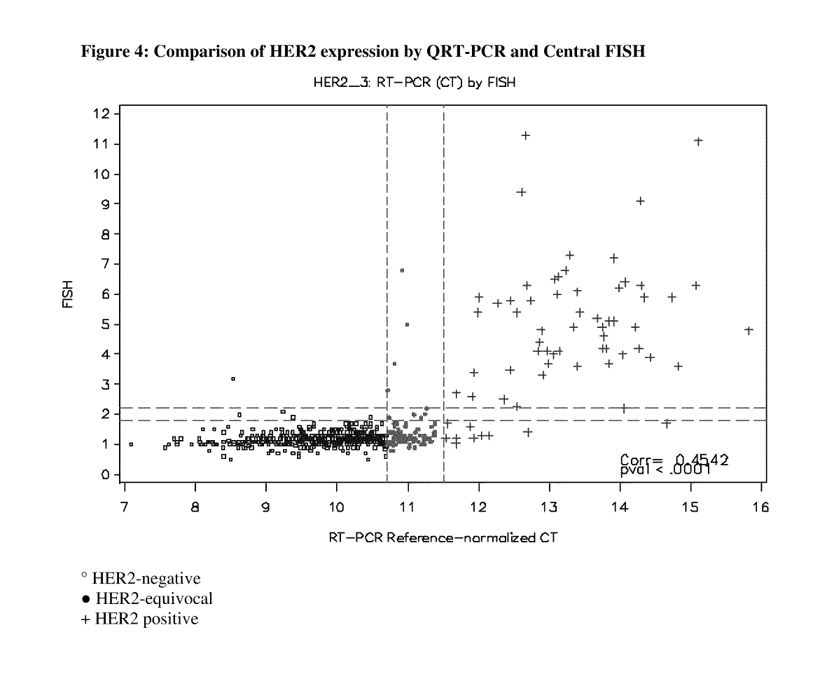

Table 4 compares HER2 status classification as determined by QRT-PCR with status classification as determined by central FISH. Table 4 includes all tumors including those which were classified by either or both QRT-PCR and central FISH as HER2-Equivocal. These results are presented in graph format in FIG. 4.

TABLE 4

| | | | Central FISH | | Central FISH | | |
|---|---|---|---|---|---|---|---|
| | Central FISH | | HER2- | | HER2- | | |
| | HER2-positive | | equivocal | | negative | | Total |
| QRT-PCR | 55 | (92%) | 1 | (10%) | 11 | (2%) | 67 |
| HER2-positive | | | | | | | |
| QRT-PCR | 4 | (7%) | 5 | (50%) | 79 | (16%) | 88 |
| HER2- | | | | | | | |
| equivocal | | | | | | | |
| QRT-PCR | 1 | (2%) | 4 | (40%) | 408 | (82%) | 413 |
| HER2- | | | | | | | |
| negative | | | | | | | |
| Total | 60 | | 10 | | 498 | | 568 |

Concordance: 82% 95% CI (79%, 85%)
Weighted Kappa: 63% 95% CI (55%, 70%)

1. A method for determining the HER2 Status of a cancer tumor sample obtained from a human patient, comprising measuring a HER2 expression level from said sample using quantitative reverse transcriptase polymerase chain reaction (QRT-PCR), normalizing the HER2 expression level relative to at least one reference gene to generate a normalized HER2 expression level, comparing the normalized HER2 expression level to predetermined HER2 expression cutpoints to determine a HER2 Status, wherein predetermined HER2 expression cutpoints are based on a validated test, wherein the HER2 Status of the cancer tumor is one of the classifications consisting of positive, negative, and equivocal, and wherein the likelihood that the sample is classified as HER2-equivocal is negatively optimized.

2. The method of claim 1, wherein the cancer tumor is a breast cancer tumor.

3. The method of claim 1, wherein negatively optimized comprises establishing the HER2 expression cutpoints such that the likelihood that the sample is classified as HER2 equivocal is one from the list consisting of less than about 5%, less than about 7.5%, less than about 10%, and less than about 17%.

4. The method of claim 1, wherein the predetermined HER2 expression cutpoints comprise an upper HER2 expression cutpoint and a lower HER2 expression cutpoint.

5. The method of claim 4, wherein if the sample is obtained from an estrogen receptor (ER)-positive breast cancer tumor, the upper HER2 expression cutpoint is used to determine whether said tumor is HER2-positive or HER2-negative.

6. The method of claim 4, wherein if the sample is obtained from an ER-negative breast cancer tumor, the lower HER2 expression cutpoint is used to determine whether said tumor is HER2-positive or HER2-negative.

7. The method of claim 1, wherein the predetermined HER2 expression cutpoints are established such that the concordance rate between HER2 Status by QRT-PCR and HER2 Status by immunohistochemistry (IHC) or fluorescence in situ hybridization (FISH) is high when measured in a statistically significant number of cancer tumors.

8. The method of claim 7, the concordance rate between HER2 Status by QRT-PCR and HER2 Status by IHC or FISH is one from the list consisting of: greater than about 95%, greater than about 92.5%, and greater than about 90%.

9. The method of claim 1, wherein the HER2 expression level is normalized relative to expression levels of ACTB, GAPDH, GUSB, RPLP0 and TFRC.

10. The method of claim 1, wherein the HER2 expression level is measured using a HER2 expression product.

11. The method of claim 10, wherein the HER2 expression product is RNA.

12. The method of claim 10, wherein the HER2 expression product is measured using QRT-PCR probe/primer sets targeted to intronic HER2 sequences.

13. The method of claim 1, wherein the normalized HER2 expression level is calculated as Standard Normalized Expression level.

14. The method of claim 1, wherein the sample is fixed paraffin embedded tissue.

15. A method to identify patients for treatment using a minimally equivocal HER2 Status of a tumor sample obtained from a human cancer patient, comprising:
measuring the expression level of a HER2 expression product in the tumor sample;
normalizing the HER2 expression level relative to at least one reference gene;
comparing the normalized expression level to predetermined HER2 expression cutpoints, wherein the predetermined HER2 expression cutpoints comprise at least a lower HER2 expression cutoff and an upper HER2 expression cutoff; and
classifying the tumor according to HER2 Status,
wherein a HER2-positive Status indicates that the patient is a candidate for treatment with a HER2 inhibitor, and
wherein a HER2-negative Status indicates the patient is unlikely to respond to treatment with a HER2 inhibitor.

16. The method of claim 15, wherein if the sample is obtained from an estrogen receptor (ER)-positive breast cancer tumor, the upper HER2 expression cutpoint is used to determine whether said tumor is HER2-positive or HER2-negative.

17. The method of claim 15, wherein if the sample is obtained from an ER-negative breast cancer tumor, the lower HER2 expression cutpoint is used to determine whether said tumor is HER2-positive or HER2-negative.

18. The method of claim 15, wherein the HER2 inhibitor is one from the groups consisting of trastuzumab and lapatanib.

19. The method of claim 15, wherein the cancer tumor is a breast cancer tumor.
